(19) United States
(12) Patent Application Publication
(10) Pub. No.: US 2004/0019357 A1
(43) Pub. Date: Jan. 29, 2004

(54) Annuloplasty ring holder

(76) Inventors: Louis A. Campbell, Austin, TX (US); Robert M. Casanova, Austin, TX (US)

Correspondence Address: WILLIAMS, MORGAN & AMERSON, P.C., 10333 RICHMOND, SUITE 1100, HOUSTON, TX, 77042, US

(21) Appl. No.: 10/205,948

(22) Filed: Jul. 26, 2002

(51) Int. Cl. A61F 11/00

(52) U.S. Cl. 606/108

(57) ABSTRACT

An annuloplasty ring holder is provided for holding an annuloplasty ring during storage and implantation. The annuloplasty ring holder includes a fixed jaw and one or more movable jaws. The movable jaw is coupled to the fixed jaw by a hinge or other means so that an annuloplasty ring may be positioned between the jaws and clamped in place. The holder is selectively coupled to a handle. The holder may be used for all types of annuloplasty rings, including full and partial rings, rigid and flexible rings, and adjustable and non-adjustable rings.

### BACKGROUND OF THE INVENTION

The present invention relates to surgical tools, namely, an apparatus for holding an annuloplasty ring during storage and implantation.

Annuloplasty rings are useful in various surgical procedures. An annuloplasty ring may be used to restore or improve the operation of a heart valve by reshaping the heart valve annulus, e.g., mitral or tricuspid annular reduction. In these types of procedures, an annuloplasty ring is positioned about a valve annulus. Sutures are passed through the valve annulus tissue and the annuloplasty ring. The sutures are tightened to hold the annuloplasty ring in a desired position about the annulus, thereby reshaping the annulus.

Depending on the condition of the valve annulus and the desired reshaping of the valve annulus, different types of annuloplasty rings may be used. An annuloplasty ring may be generally rigid or flexible, full or partial, and adjustable or non-adjustable. In working with any of these types of annuloplasty rings, a surgeon needs a mechanism to securely hold the annuloplasty ring in position while the surgeon places sutures in the ring. Any such mechanism should provide the surgeon with good visual and manual access to the annulus area for proper suturing.

One approach for holding an annuloplasty ring includes positioning the ring in a groove of a holder, see, e.g., U.S. Pat. No. 5,350,420, incorporated herein by reference. In this approach, the annuloplasty ring is positioned in the groove and held in place by sutures internal to the annuloplasty ring. In the case of a full annuloplasty ring, the ring must be stretched over the groove lip to be positioned in the groove. Such stretching may damage the ring. Also, an annuloplasty ring is typically coupled with its holder during the manufacturing process. Therefore, the ring and holder may be in storage for many months or even years. The sutures used to hold the ring in the groove must be biocompatible and are typically made from polyester or PTFE. Also, the sutures are under constant tension. Over time, these types of sutures may stretch or “creep”. At some point, these sutures may no longer securely hold the ring in the holder groove.

In another approach for holding an annuloplasty ring, a ring is positioned internal to a series of fingers extending radially from a central base, see, e.g., U.S. Pat. No. 5,011,481, incorporated herein by reference. The ring is he sutures passing through the ring. Again, the sutures used to hold the ring may stretch over time, and may no longer securely hold the ring adjacent the fingers. Therefore, there is a need for an annuloplasty ring holder that will securely hold the ring for a prolonged period of time.

As described above, an annuloplasty ring is sutured to a valve annulus during implantation. With adjustable rings, the adjustment mechanism, e.g., one or more draw strings, is internal to the ring. Therefore, the surgeon cannot see the internal adjustment mechanism. However, it is crucial that the attaching sutures are not tangled with the ring-adjustment mechanism to ensure that the adjustment mechanism properly functions. Consequently, there is a need for a ring holder that protects the adjustment mechanism during the suturing process.

### SUMMARY OF THE INVENTION

The present invention is directed toward a holder for annuloplasty rings. The holder includes a fixed jaw member and a movable jaw member. The jaw members are operably coupled so that the movable jaw member is selectively movable relative to the fixed jaw member. In one embodiment, the coupling mechanism includes a pin extending into both the fixed jaw member and movable jaw member, forming a hinge. Means are provided for selectively actuating the movement of the movable jaw member relative to the fixed jaw member. Additionally, a locking mechanism is provided for selectively locking the position of the movable jaw member relative to the fixed jaw member.

A handle is selectively coupled with the fixed jaw member. In one embodiment, the handle is axially aligned with the jaw members. In another embodiment, the handle is radially aligned with the jaw members.

In one embodiment, the fixed jaw member includes a base and a peripheral edge spaced radially outward from the base. In another embodiment, the fixed jaw member includes a base and a plurality of fingers extending radially outward from the base. In another embodiment, the movable jaw member includes a plurality of fingers.

The present invention provides several advantages. First, it provides a holder with good visual and manual access to the implantation area. Also, the clamping mechanism securely holds the ring for an extended period of time without stretching the ring or suturing the ring to a holder. Also, the clamping mechanism provides quick and easy release of the ring. In the case of adjustable rings, the clamping mechanism serves to protect the adjustment mechanism internal to the ring. Finally, the present invention is useful for many types of annuloplasty rings.

### BRIEF DESCRIPTION OF THE DRAWINGS

FIG. 1 is a side view of one embodiment of an annuloplasty ring holder;

FIG. 2 is a side view of another embodiment of an annuloplasty ring holder;

FIG. 3 is a top view of the embodiment of FIG. 2;

FIG. 4 is a side view of another embodiment of an annuloplasty ring holder;

FIG. 4a is a side view of another embodiment of an annuloplasty ring holder;

FIG. 4b is a front view of another embodiment of an annuloplasty ring holder;

FIG. 5 is a top view of another embodiment of an annuloplasty ring holder; and

FIG. 5a is a side view of the embodiment of FIG. 5.

### DETAILED DESCRIPTION OF THE EMBODIMENTS

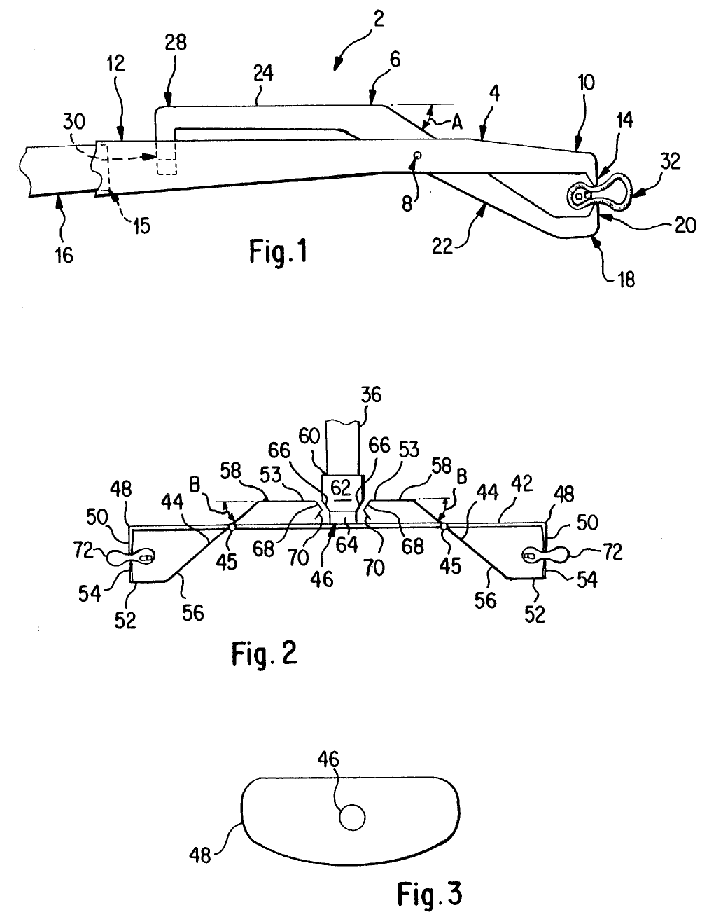

One embodiment of the invention is shown in FIG. 1. This embodiment is particularly useful for holding partial annuloplasty rings. Ring holder 2 includes fixed jaw member 4 and movable jaw member 6. Movable jaw member 6 is connected with fixed jaw member 4 by pin 8, forming a hinge, which allows movable jaw member 6 to pivot about pin 8. Other coupling mechanisms may also be used which allow movable jaw member 6 to move relative to fixed jaw member 4.

Fixed jaw member 4 includes a first, clamp end 10 and a second end 12. A clamp tooth or lip 14 extends generally orthogonal from first end 10. Second end 12 includes a receiving mechanism 15 for selectively receiving a handle 16 or other device useful for positioning holder 2. As shown in the embodiment of FIG. 1, handle 16 extends radially along the major axis of holder 2. In one embodiment, receiving mechanism 15 includes a threaded bore positioned in second end 12 (as shown in FIG. 1) which receives a threaded end of handle 16. Other coupling mechanisms may also be used.

Movable jaw member 6 also includes a first, clamp end 18. A clamp tooth or lip 20 extends generally orthogonal from first end 18 and toward clamp tooth 14. Movable jaw member further includes first arm section 22 and second arm section 24, where second arm section 24 deflects from first arm section 22 by angle A. The length of arm sections 22, 24 and the size of angle A are selected so that first end 18 moves in a smooth arcuate motion relative to first end 10 and so that tooth 20 is generally aligned with tooth 14 when first end 18 is moved toward first end 10.

Movable jaw member 6 further includes a second end 28 coupled to second arm section 24. Second end 28 of movable jaw member 6 includes coupling member 30 for selectively coupling movable jaw member 6 in a fixed position relative to fixed jaw member 4. As shown in the embodiment of FIG. 1, coupling member 30 extends generally orthogonal from second arm section 24 and toward fixed jaw member 4. In one embodiment, fixed jaw member 4 includes a bore for receiving coupling member 30. In another embodiment, coupling member 30 includes one or more raised surfaces, which slide over raised surfaces on fixed jaw member 4 and lock movable jaw member 6 in place. Other types of coupling mechanisms may also be used to selectively lock movable jaw member 6 in a fixed position relative to fixed jaw member 4.

As shown in FIG. 1, an annuloplasty ring 32 is positioned between teeth 14 and 20. By moving the second end 28 of movable jaw member 6 toward fixed jaw member 4, teeth 14 and 20 pinch or clamp down on ring 32, securely holding ring 32 to holder 2. In the case of an adjustable ring, the teeth 14, 20 are positioned so that a suture portion of the ring is exposed for suturing to the valve annulus, while the adjustment mechanism is protected from the suturing process.

Another embodiment of the invention is shown in FIG. 2. The clamping mechanism is similar to that shown in FIG. 1, however, handle 36 is positioned axially relative to the clamping mechanism, as opposed to the radial alignment of FIG. 1. The axial alignment of the handle is useful for holders that can be used with both full and partial rings.

Ring holder 40 includes fixed jaw member 42 coupled with one or more movable jaw members 44. In the embodiment of FIG. 2, two movable jaw members 44 are provided. In other embodiments, three or more movable jaw members 44 are provided. Such a configuration may be particularly useful with annuloplasty rings that are rigid for more than 180 degrees. Movable jaw members 44 are coupled with fixed jaw member 42 by pins 45, forming a hinge as described above. Other coupling mechanisms may also be used which allow selective movement of movable jaw members 44 relative to fixed jaw member 42.

Fixed jaw member 42 includes a central base 46 and radially spaced, peripheral edge 48. Clamp tooth 50 extends generally orthogonal from clamp edge 48. In the embodiment of FIG. 3, peripheral edge 48 is generally in the shape of a AD@, i.e., the general shape of a typical full mitral annuloplasty ring. In other embodiments, the holder may be of a shape suitable for the specific application, e.g., more round or oval shaped.

Referring again to FIG. 2, movable jaw members 44 include first clamp ends 52 and second ends 53. Clamp teeth 54 extend generally orthogonal from clamp ends 52 and toward clamp tooth 50. Movable jaw members 44 further include first arm sections 56 and second arm sections 58, where second arm sections 58 deflect from first arm sections 56 by angle B. The length of arm sections 56, 58 and the size of angle B are selected so that first ends 52 move in a smooth arcuate motion relative to edge 48 and so that teeth 54 are generally aligned with tooth 50 when first ends 52 are moved toward edge 48.

Jaw actuating mechanism 60 is coupled with base 46. In the embodiment of FIG. 2, jaw actuating mechanism 60 includes threaded nut 62 positioned on threaded mount 64. Nut 62 includes camming surfaces 66. Second ends 53 of movable jaw members 44 include first camming surfaces 68 and second camming surfaces 70. By rotating nut 62 in a first direction, nut 62 moves toward base 46 and camming surfaces 66 contact first camming surfaces 68, thereby forcing second ends 53 toward base 46 and first ends 52 toward edge 48. This provides a secure clamp of an annuloplasty ring 72 between tooth 50 and teeth 54, as shown in FIG. 2. Consequently, nut 62 also serves as a locking mechanism to hold ring 72 in place.

As in the embodiment of FIG. 1, an adjustable ring may be positioned between tooth 50 and teeth 54 so that the adjustment mechanism internal to ring 72 is protected from suturing.

By rotating nut 62 in the opposite direction, camming surfaces 66 move away from first camming surfaces 68 and teeth 54 move away from tooth 50, thereby releasing ring 72. Again, this clamping mechanism will hold an annuloplasty ring for a long period of time without stretching or otherwise degrading the strength of the attachment to holder 40. Other jaw actuating mechanisms may also be used.

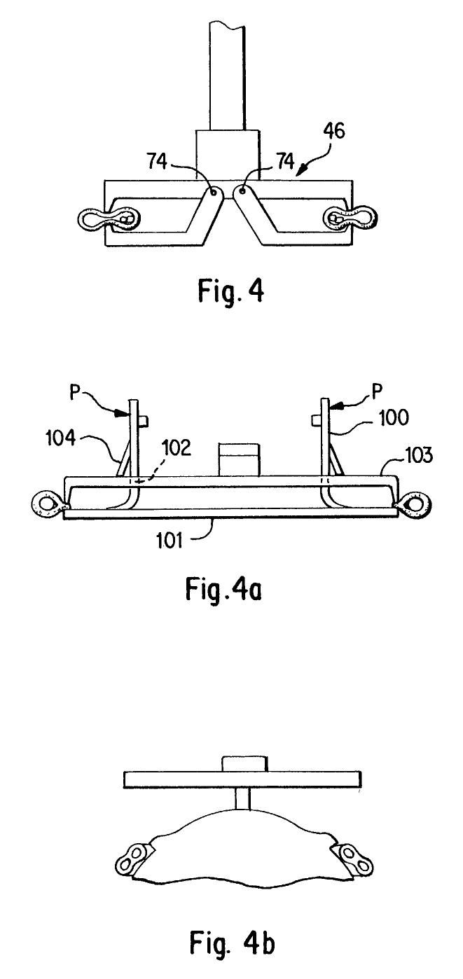

Another embodiment of the invention is shown in FIGS. 4, 4a and 4b. This embodiment is similar to FIG. 2; however, pins 74 are positioned centrally on base 46. The jaw can be held in place by a loop of suture that would be cut by the implanting surgeon. Alternatively, as shown in FIG. 4a, a cantilevered tab 100 attached to the distal jaw 101 could protrude through a rectangular hole 102 in the proximal jaw 103, engaging the proximal jaw with a tab 104 that can be released by the surgeon applying finger pressure P to the cantilevered tab 100.

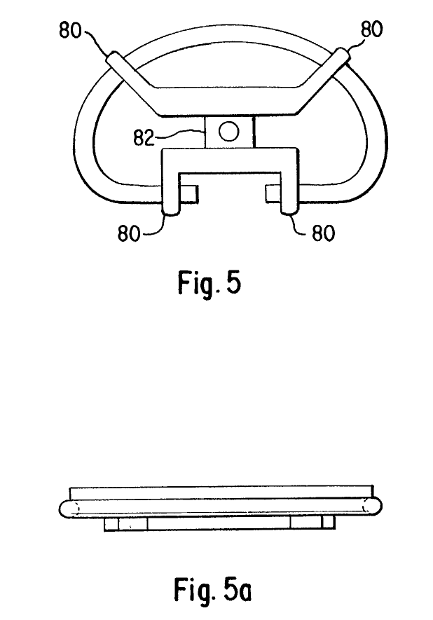

Another embodiment of the invention is shown in FIGS. 5 and 5a. In this embodiment, four fingers 80 extend from base 82 to form a fixed jaw member. A movable jaw member (not shown) includes four fingers generally aligned with fingers 80. As described above, the movable jaw member fingers are selectively movable relative to the fixed jaw member fingers, providing a clamping mechanism to securely hold an annuloplasty ring. Other embodiments of the invention may include more or fewer fingers for both the fixed and movable jaw members.

Other embodiments are within the scope of the following claims.

We claim the following:

1. An apparatus for holding an annuloplasty ring, comprising:
a fixed jaw member having a clamping end;
a movable jaw member having a clamping end and being movable relative to said clamping end of said fixed jaw member for clamping an annuloplasty ring in compression for implantation in a heart valve annulus; and
a coupling for operably connecting the fixed and movable jaw members to provide releasable compression clamping for holding the annuloplasty ring.

2. The apparatus of claim 1, further comprising locking means for selectively locking the position of the movable jaw member relative to the fixed jaw member.

3. The apparatus of claim 1, further comprising a handle selectively coupled with the fixed jaw member.

4. The apparatus of claim 3, wherein the handle is axially aligned with said fixed jaw member.

5. The apparatus of claim 3, wherein the handle is radially aligned with said fixed jaw member.

6. The apparatus of claim 1, wherein the coupling means includes a pin extending into both the fixed jaw member and movable jaw member, forming a hinge.

7. The apparatus of claim 1, wherein the fixed jaw member includes a base and the peripheral edge is spaced radially outward from the base.

8. The apparatus of claim 1, wherein the fixed jaw member includes a base and a plurality of fingers extending radially outward from the base.

9. The apparatus of claim 1, wherein the movable jaw member includes a plurality of fingers.

10. The apparatus of claim 1, further comprising means for selectively actuating the movement of the movable jaw member relative to the fixed jaw member.

11. An apparatus for holding an annuloplasty ring, comprising:
a first jaw member having a clamping end; and
a second jaw member having a clamping end and being operably coupled to said first jaw member, said second jaw member being movable relative to said first jaw member to provide a clamp for holding an annuloplasty ring in compression between said first and said second jaw members.

12. The apparatus of claim 11 wherein said first and said second jaw members further comprise a plurality of fingers which cooperate to clamp said annuloplasty ring at a plurality of locations along said ring.
